(19) United States
(12) Patent Application Publication
(10) Pub. No.: US 2013/0190737 A1
(43) Pub. Date: Jul. 25, 2013

(54) APPLICATION OF ENERGY IN MEDICAL TREATMENTS

(71) Applicants: David Muller, Boston, MA (US); John Marshall, Farnborough, Hants (GB)

(72) Inventors: David Muller, Boston, MA (US); John Marshall, Farnborough, Hants (GB)

(21) Appl. No.: 13/738,675

(22) Filed: Jan. 10, 2013

Related U.S. Application Data
(60) Provisional application No. 61/584,916, filed on Jan. 10, 2012.

(51) Int. Cl. A61F 9/007

(52) U.S. Cl. CPC A61F 9/0079; A61F 9/00736
USPC 606/6

(57) ABSTRACT

Embodiments apply light energy in medical treatments. To enhance or control the effect of the light energy, embodiments apply the light energy after tissue has been treated, e.g., with a photosensitizing agent. For example, embodiments may treat target tissue with riboflavin before exposure to ultraviolet light. For example, a system for cataract surgery includes a removal system configured to remove a first lens from an eye, wherein a capsular bag remains in the eye after removal of the first lens. The system includes an application system configured to treat lenticular epithelial cells in the capsular bag with the photosensitizing agent. The system includes a delivery system with a light source and an optical device. The optical device delivers light to the treated lenticular epithelial cells. Energy from the light destroys the lenticular epithelial cells in the capsular bag to reduce the growth of epithelial cells that cause posterior capsule opacification.

### CROSS REFERENCE TO RELATED APPLICATIONS

This application claims priority to U.S. Provisional Patent Application No. 61/584,916, filed Jan. 10, 2012, the contents of which are incorporated entirely herein by reference.

### BACKGROUND OF THE INVENTION

1. Field of the Invention

The invention pertains to the field of medical treatment and, more particularly, to the application of energy in medical treatments, for example, the application of ultraviolet light in combination with riboflavin to destroy pathogens and/or undesired cells.

2. Description of Related Art

Cataract surgery is one of a number of surgical procedures for treating various eye disorders. A cataract develops when the crystalline lens of the eye experiences opacification due to metabolic changes that occur in the crystalline lens fibers over time. During cataract surgery, this crystalline lens is extracted from the capsular bag and replaced with an artificial intraocular lens that restores transparency to the lens in the eye. The invasive nature of cataract surgery increases the risk of infection. In particular, endophthalmitis is a rare but serious complication associated with cataract surgery, where internal linings in the intraocular portions of the eye suffer from inflammation. Endophthalmitis is often caused by bacterial or fungal infection. Moreover, cataract surgery may be accompanied by other complications such as posterior capsule opacification (PCO), which occurs when the growth of lenticular epithelial cells remaining in the capsular bag after extraction of the crystalline lens causes haziness that results in blurry vision.

Other areas of the human body are also susceptible to bacterial or fungal infection. In particular, areas of the body having an orifice or cavity may be prone to certain types of infection. For example, bladder infections occur when bacteria find their way into the bladder. In another example, infection in the stomach may be caused by Helicobacter pylori (H. pylori) bacteria. Acute infection with H. pylori may manifest itself with acute gastritis with abdominal pain and nausea. Chronic gastritis may develop with abdominal pain, nausea, bloating, belching, and/or vomiting. Individuals infected with H. pylori have a 10 to 20% lifetime risk of developing peptic ulcers and a 1 to 2% risk of acquiring stomach cancer.

### SUMMARY

In general, embodiments according to aspects of the present invention apply light energy in medical treatments. To enhance or otherwise control the effect of the light energy, embodiments apply the light energy to tissue after the tissue has been treated, e.g., with a photosensitizing agent. For example, embodiments may treat target tissue with riboflavin before exposing the target tissue to ultraviolet (UV) light. In particular, embodiments apply light energy, e.g., UV light, to destroy pathogens and/or undesired cells during medical treatments.

According to one embodiment, a method for cataract surgery includes removing a first lens from an eye, wherein a capsular bag remains in the eye after removal of the first lens. The method also includes treating lenticular epithelial cells in the capsular bag with a photosensitizing agent, and delivering light to the treated lenticular epithelial cells. Energy from the light destroys the lenticular epithelial cells in the capsular bag to reduce the growth of epithelial cells that cause posterior capsule opacification after cataract surgery. The method also includes implanting a second lens in the capsular bag to replace the removed first lens.

According to another embodiment, a system for cataract surgery includes a removal system configured to remove a first lens from an eye, wherein a capsular bag remains in the eye after removal of the first lens. The system also includes an application system configured to treat lenticular epithelial cells in the capsular bag with the photosensitizing agent. Furthermore, the system includes a delivery system with a light source and an optical device. The optical device delivers light generated from the light source to the treated lenticular epithelial cells. Energy from the light destroys the lenticular epithelial cells in the capsular bag to reduce the growth of epithelial cells that cause posterior capsule opacification after cataract surgery.

In the embodiments above, the light may be ultraviolet (UV) light and the photosensitizing agent may be riboflavin. The light may be delivered to the lenticular epithelial cells according to multiphoton excitation. The light may be delivered to the lenticular epithelial cells includes via an optical device that is positioned external to the eye and directs the light to the capsular bag. Alternatively, the light may be delivered to the lenticular epithelial cells includes via an optical device that is positioned internally in the capsular bag, the light being transmitted outwardly from the optical device.

Additional aspects of the invention will be apparent to those of ordinary skill in the art in view of the detailed description of various embodiments, which is made with reference to the drawings, a brief description of which is provided below.

### BRIEF DESCRIPTION OF THE DRAWINGS

FIG. 1 illustrates an example approach for sterilizing a field prior to treatment according to aspects of the present invention.

FIG. 2 illustrates an example approach for sterilizing aspects of an eye during cataract surgery according to aspects of the present invention.

FIG. 3 illustrates an example approach for destroying lenticular epithelial cells in the capsular bag during cataract surgery according to aspects of the present invention.

FIG. 4 illustrates an example device for delivering UV light to portions of the eye treated with riboflavin according to aspects of the present invention.

FIG. 5A illustrates an example device for delivering UV light to a body part treated with riboflavin according to aspects of the present invention.

FIG. 5B illustrates another example device for delivering UV light to a body part treated with riboflavin according to aspects of the present invention.

FIG. 6 illustrates another example approach for cataract surgery, including destroying lenticular epithelial cells in the capsular bag, according to aspects of the present invention.

FIG. 7A illustrates an example approach for treating bacteria in the bladder according to aspects of the present invention.

FIG. 7B illustrates an example approach for treating bacteria in the stomach according to aspects of the present invention.

### DESCRIPTION

In general, embodiments according to aspects of the present invention apply light energy in medical treatments. To enhance or otherwise control the effect of the light energy, embodiments apply the light energy to tissue after the tissue has been treated, e.g., with a photosensitizing agent. For example, embodiments may treat target tissue with riboflavin before exposing the target tissue to ultraviolet (UV) light, i.e., light having a wavelength of approximately 10 nm to approximately 400 nm and corresponding photon energies from approximately 3 eV to approximately 124 eV. In particular, embodiments apply light energy, e.g., UV light, to destroy pathogens and/or undesired cells during medical treatments.

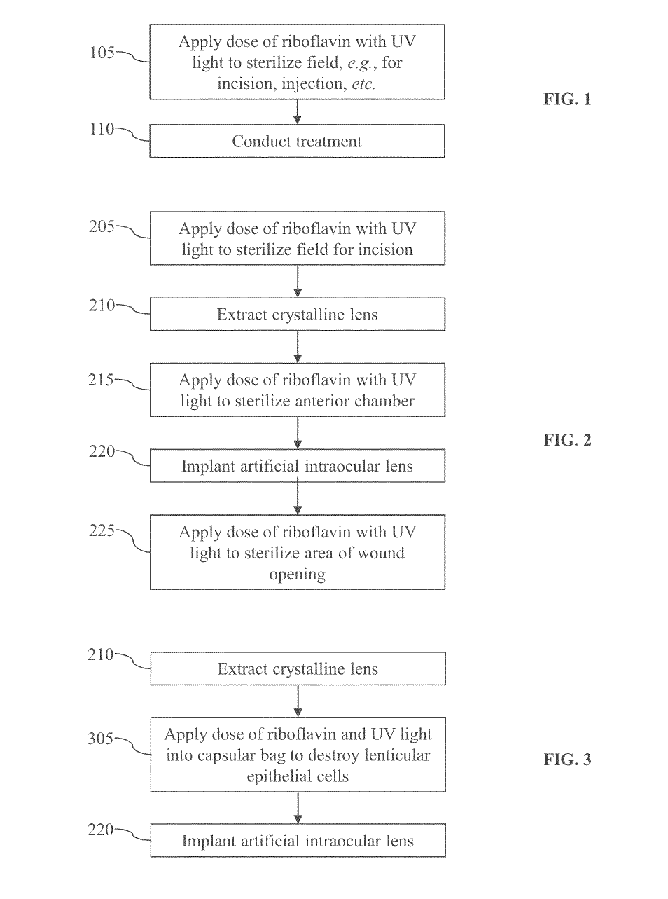

In some cases, the combined application of riboflavin and UV light provides a light-based sterilizing effect that reduces the risk of infection associated with the medical treatments. Referring to FIG. 1, an example embodiment prophylactically applies in vivo a combination of riboflavin and ultraviolet (UV) light in act 105, respectively, to sterilize a treatment field prior to a corresponding medical treatment in act 110. There may be a soak time for the riboflavin prior to the delivery of the UV light to allow proper doses of riboflavin to reach all areas of the treatment field. The medical treatment in act 110, for example, may involve incision, injection, or other penetration of the outer protective layer of the body. The amount of riboflavin and the exposure to the UV light is sufficient to achieve sterility in the field while minimizing any damage or other unwanted effects in the tissue. Embodiments control aspects of the delivery of both the riboflavin and/or the UV light.

According to one mechanism, the riboflavin acts as a photosensitizer that increases the absorption of UV light. The resulting absorption of UV light can induce DNA and RNA lesions, and as a result, is effective in killing viruses, bacteria, and other pathogens in the field. Thus, the effect of applying the UV light energy is promoted and/or otherwise controlled by employing riboflavin as a photosensitizing agent. According to an additional or alternative mechanism, the UV light excites riboflavin and causes the riboflavin to react with oxygen to form singlet molecular oxygen and/or other radicals. The singlet molecular oxygen and/or other radicals may also act to sterilize the treated areas.

Cataract surgery is an example of an invasive treatment that may increase the risk of infection. In particular, endophthalmitis is a rare but serious complication associated with cataract surgery, where bacterial or fungal infection may cause internal linings in the intraocular portions of the eye to suffer from inflammation.

Accordingly, referring to the example embodiment of FIG. 2, UV light is employed with the photosensitizing effects of riboflavin to sterilize aspects of the eye during cataract surgery and reduce the risk of infection. In act 205, riboflavin and then UV light may be applied on the exterior to sterilize the field prior to incision. In act 210, the crystalline lens with the cataract is extracted. In act 215, riboflavin is introduced into the anterior chamber of the eye and the anterior chamber irradiated through the cornea with UV light to sterilize the aqueous humor in the anterior chamber. Additional action may be taken to ensure that the corneal endothelium is protected, e.g., by applying a blocking agent, such as hyaluronic acid, to protect the endothelium from the riboflavin. In act 220, an artificial intraocular lens is implanted to replace the crystalline lens. In act 225, the area of the wound opening from incision may also be sterilized with the combination of riboflavin and UV light.

Accordingly, FIG. 2 shows that the combination of riboflavin and UV light may be employed to sterilize various aspects, e.g., the intraocular portions, of the eye during an eye treatment. Advantageously, the sterilization acts during cataract surgery as shown in FIG. 2 reduce the risk of infection and complications such as endophthalmitis. In general, similar sterilization steps employing riboflavin and UV light may be applied in any treatment involving the eye.

A solution containing riboflavin may be applied to a treatment field in single, continuous, or intermittent doses. In some cases, the application of riboflavin solution may involve spraying a treatment field, e.g., an exterior area around an incision. In other cases, portions of a cavity may be filled at least partially with a riboflavin solution to treat the interior areas with sufficient doses of riboflavin. A syringe, for example, filled with the riboflavin solution may be employed to deliver riboflavin into the cavity.

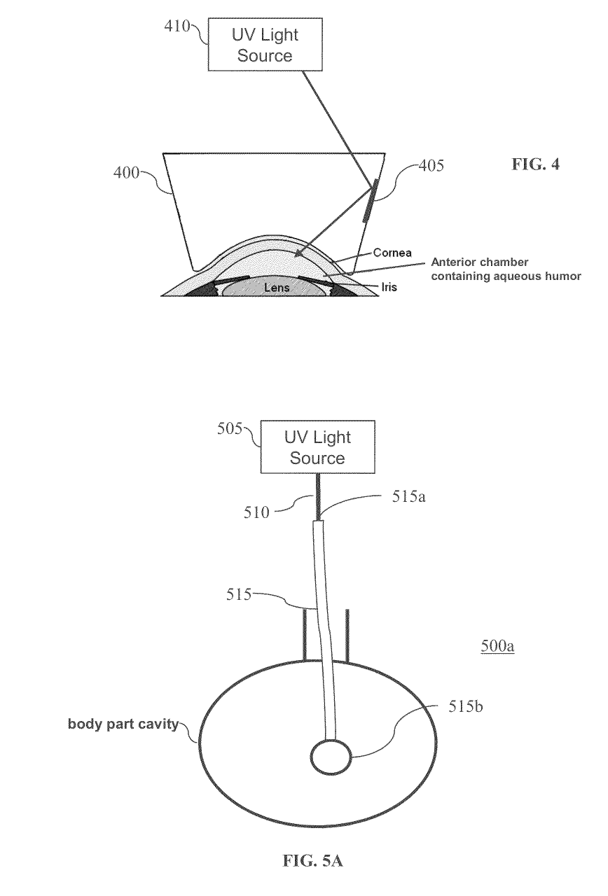

In some embodiments, the irradiation of the riboflavin in the sterilizing acts above may be achieved with a device similar to a goniolens that appropriately directs light from a UV light source and enables irradiation of large areas of the intraocular space. According to aspects of the present invention, an optical device 400 as shown in FIG. 4 includes a configuration of one or more mirrors 405, which direct irradiating light from a UV light source 410 to the areas treated with riboflavin, e.g., the anterior chamber. The UV light source 410 can be operated to deliver different wavelengths of light at selected times.

The application of riboflavin and UV light is not limited to sterilizing a treatment field. In some cases, UV light can also be applied with riboflavin to destroy undesired cells and/or reduce the growth of undesired cells. Indeed, such an application can also be illustrated in the context of cataract surgery. The cataract treatment shown in FIG. 3 may include the acts shown in FIG. 2. FIG. 3, however, also includes an additional act 305 that applies a sufficient dose of riboflavin and UV light to destroy the lenticular epithelial cells that line the capsular bag. The capsular bag is the structure remaining within the eye following the cataract extraction in act 210. The implanted intraocular lens is placed within the capsular bag to recreate the usual phakic state. Destroying lenticular epithelial cells in the capsular bag in act 305 during the cataract surgery is advantageous, because it reduces the risk of posterior capsule opacification after surgery. Posterior capsule opacification (PCO) is a common post-operative complication of cataract surgery, where the growth of lenticular epithelial cells remaining in the capsular bag causes haziness with the capsular bag resulting in blurry vision. Destroying the lenticular epithelial cells reduces this unwanted growth.

The sterilization acts described with reference to FIG. 2 may be additionally applied in the treatment of FIG. 3. The dose of riboflavin and the corresponding amount of UV light in act 305 may be different from the sterilizing acts. However, the result of act 305 may also result in sterilizing the interior of the capsular bag prior to the implantation of the intraocular lens in act 220.

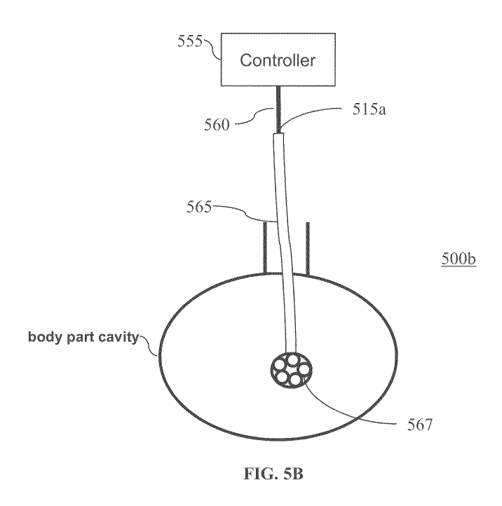

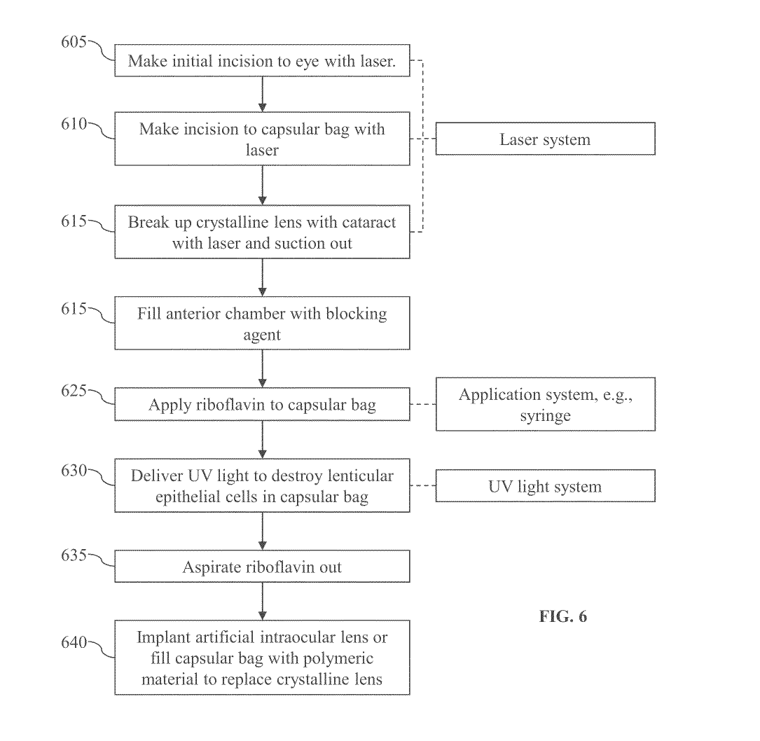

FIG. 5A illustrates an example irradiating system 500a that may be employed to irradiate the interior of the capsular bag in act 305 shown in FIG. 3. The system 500a includes a UV light source 505, an optical fiber 510, and a three-dimensional light-transmitting optical device 515. The UV light source 505 can be operated to deliver different wavelengths of light at selected times. A first end 515a of the light-transmitting optical device 515 is coupled to the UV light source 505 via the optical fiber 510. UV light generated by the source 505 is transmitted to the light-transmitting optical device 515 and is then transmitted outwardly from the light-transmitting optical device 515 in many directions. The second end 515b of the light-transmitting optical device 515 is configured, e.g., sized and dimensioned, to be inserted into the capsular bag after the crystalline lens has been extracted in act 210. As shown in FIG. 5, the second end 515b of the light-transmitting optical device 515 is configured as a ball diffuser, where the UV light is transmitted radially outward in substantially all directions from the second end 515b. As such, when inserted into the capsular bag treated with riboflavin, the light-transmitting optical device 515 can deliver UV light to substantially all areas inside the capsular bag without a significant amount of operation. It is understood that the shape of the second end 515b of the light-transmitting optical device 515 is not limited to a substantially spherical shape. The light-transmitting optical device 515 may employ any shape that delivers UV light in many directions and sufficiently irradiates the desired interior areas of the capsular bag. For example, the second end 515b may be similar to the natural shape of the capsular bag or may be shaped similar to the intraocular lens that is implanted in the capsular bag.

Some embodiments may deliver the UV light according to single photon excitation, which involves applying photons of a particular wavelength and corresponding photon energy to the target tissue. Other embodiments, however, may control the irradiating light by employing aspects of multiphoton excitation microscopy. In particular, rather than delivering a single photon of a particular wavelength and higher corresponding photon energy to irradiate the body part, the irradiating system delivers multiple photons of longer wavelengths, i.e., lower energy, that work in combination with the photosensitizing effects of the riboflavin. Advantageously, longer wavelengths are absorbed and scattered to a lesser degree than shorter wavelengths. For example, in some embodiments, two photon energies may be employed, where each photon carries approximately half the energy necessary to irradiate the body part with the desired energy. The probability of the near-simultaneous absorption of multiple photons is low, so a high flux of excitation photons is typically required. Because multiple photons are absorbed during the irradiation, the probability for delivery of the desired energy level increases with intensity. Controlling the intensity with multiphoton excitation advantageously allows for more control over the delivery of the energy with the UV light. Such control enhances the safety of the systems and methods described herein. For example, in some embodiments, multiple wavelengths of UV light with lower energies can then be transmitted through the cornea, e.g., via the optical device 400 and the light source 410, and absorbed only at the lenticular epithelial cells that line the capsular bag, thereby minimizing any damage or other unwanted effects in other aspects of the eye.

Referring to FIG. 6, aspects of the present invention are further illustrated in another example application during cataract surgery. In act 605, an initial incision is made in the eye. In act 610, a further incision is made in the capsular bag. The incisions may be achieved by applying a laser with sufficient and controlled energy to incise the tissue. In act 615, the crystalline lens with the cataract is broken up with the laser and suctioned out, e.g. though a small 2 mm diameter hole. In act 620, the anterior chamber is filled with a blocking agent, such as hyaluronic acid, to protect the endothelium from the riboflavin. In act 625, the riboflavin is applied to the capsular bag. In act 630, a light-transmitting optical device (e.g., the light-transmitting optical device 515 as shown in FIG. 5) is inserted into the eye and manipulated along the capsular bag wall to destroy the lenticular epithelial cells with UV light. Alternatively, another optical technique is employed to deliver UV light to the riboflavin applied along the capsular bag wall according to multiphoton excitation to kill the lenticular epithelial cells as described above. In act 635, the riboflavin is aspirated out. In act 640, an artificial intraocular lens is implanted to replace the crystalline lens. Alternatively, in act 640, the capsular bag is filled with a polymeric material that retains pliability and provides some accommodation when it polymerizes to replace the crystalline lens. The polymeric material may be set by applying UV light through the cornea, e.g., through the optical device 100 shown in FIG. 4. When the polymeric material remains unset, the small incision in the capsular bag can be sealed with heat or a light activated bonding material to prevent the polymeric material from leaking from the capsular bag. During the embodiment illustrated in FIG. 6, riboflavin and UV light may be applied as one or more additional sterilizing steps as described with reference to FIG. 2. The example of FIG. 6 may employ a multifunctional light system that provides the laser as well as the UV light source.

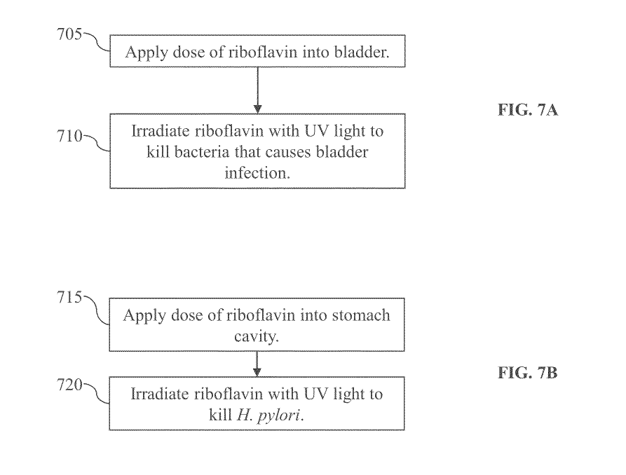

Although the embodiments described above may involve invasive treatments for the eye, riboflavin and UV light may be applied in treatments of other aspects of the body, e.g., to sterilize a field for any invasive or non-invasive procedure on other aspects of the body. For example, referring to the embodiment of FIG. 7A, riboflavin is introduced into the bladder in act 705 and subsequently irradiated with UV light in act 710 to kill bacteria that cause bladder infection. Meanwhile, in the embodiment of FIG. 7B, riboflavin is introduced into the stomach in act 715 and subsequently irradiated with UV light in act 720 to kill H. pylori bacteria, which may cause acute infection.

Accordingly, irradiating systems similar to that shown in FIG. 5 may be employed to irradiate the interior of other body cavities treated with riboflavin. In general, aspects of the present invention contemplate inserting a light-transmitting optical device that transmits UV light in desired directions within any type of body cavity. In the examples of FIGS. 7A and 7B, a light-transmitting optical device, e.g., configured as a ball diffuser, may be inserted into the bladder or stomach.

FIG. 5B illustrates another example irradiating system 500b that may also be employed to irradiate the interior of a body cavity corresponding, for example, to the bladder or stomach. The irradiating system 500b includes a controller 555 and a light-emitting optical device 565, which may be electrically coupled by one or more wires 560. Rather than generating the UV light at a distal light source and irradiating the cavity interior via a light-transmitting optical device, the irradiating system 500b employs a light-emitting optical device 565 that generates the UV light within the cavity. For example, the light-emitting optical device 565 may include one or more UV light sources 567, e.g., light emitting diodes (LED's), which are positioned in the cavity during operation of the irradiating system 500b. The UV light sources 567 are arranged to deliver UV light in different directions within the cavity to targeted areas. The controller 555 is operated to deliver power to and activate the UV light sources 567. In some cases, the controller 555 may be operated to activate the UV light sources 567 selectively, i.e., where some UV light sources 567 are activated to deliver light in specific directions while the other UV light sources 567 remain inactive. Additionally, the UV light sources 567 may provide light of varying wavelengths, so that the controller 555 may be operated to activate the UV light sources 567 selectively according to the desired wavelength(s).

As FIG. 5B illustrates, aspects of the present invention may employ a controller to manage aspects of the operation delivery of riboflavin and UV light. In some embodiments, the controller may include computer processing hardware that receives program instructions from a computer-readable storage medium to provide signals, data, etc., to various subsystems of the embodiments to execute any of the processes described herein. In some cases, the controller may be communicatively coupled to the monitoring system to process the images, data, etc., from the monitoring system and to determine a response. For example, in some embodiments, the monitoring system includes an optical coherence tomography (OCT) system or other imaging system to generate an image, e.g., a three-dimensional image, of the target tissue, which can be evaluated to determine the effect of the treatment, e.g., the destruction of lenticular epithelial cells. Such monitoring systems can be inserted, for example, in a body cavity to generate feedback signals. The monitoring systems may also be integrated in some aspect with the systems that direct the UV light to the target tissue.

Embodiments according to aspects of the present invention contemplate more general approaches for applying energy to provide sterilization or other advantages, e.g., killing lenticular epithelial cells in the capsular bag, during medical treatment, e.g., cataract surgery. The combined application of riboflavin and UV light in medical treatments is merely an example of an advantageous application of energy in the form of light, where the riboflavin acts as a photosensitizer for the UV light and/or becomes excited by the UV light to provide the desired results. Furthermore, it is understood that the light applied according to aspects of the present invention is not limited to ultraviolet wavelengths and that other wavelengths of light can provide sufficient energy.

In general, while aspects of the present invention have been described in connection with a number of exemplary embodiments, and implementations, the present inventions are not so limited, but rather cover various modifications, and equivalent arrangements. Any combination(s) of the features described in any of the embodiments described herein may be the subject of claims for the present application.

What is claimed is:

1. A method for cataract surgery, comprising:
removing a first lens from an eye, wherein a capsular bag remains in the eye after removal of the first lens;
treating lenticular epithelial cells in the capsular bag with a photosensitizing agent;
delivering light to the treated lenticular epithelial cells, wherein energy from the light destroys the lenticular epithelial cells in the capsular bag to reduce the growth of epithelial cells that cause posterior capsule opacification after cataract surgery; and
implanting a second lens in the capsular bag to replace the removed first lens,

2. The method of claim 1, wherein the light is ultraviolet (UV) light and the photosensitizing agent is riboflavin.

3. The method of claim 1, wherein delivering the light to the lenticular epithelial cells includes delivering the light according to multiphoton excitation.

4. The method of claim 1, wherein delivering the light to the lenticular epithelial cells includes delivering the light via an optical device positioned external to the eye, the optical device directing the light to the capsular bag.

5. The method of claim 1, wherein delivering the light to the lenticular epithelial cells includes delivering the light via an optical device positioned internally in the capsular bag, the light being transmitted outwardly from the optical device.

6. The method of claim 1, further comprising making an incision in the eye and the capsular bag with a laser, wherein removing the first lens of the eye includes breaking up the first lens with the laser.

7. The method of claim 1, wherein implanting a second lens includes filling the capsular bag with a polymeric material.

8. A system for cataract surgery, comprising:
a removal system configured to remove a first lens from an eye, wherein a capsular bag remains in the eye after removal of the first lens;
an application system including a photosensitizing agent, the application system being configured to treat lenticular epithelial cells in the capsular bag with the photosensitizing agent; and
a delivery system including a light source and an optical device, the optical device delivers light generated from the light source to the treated lenticular epithelial cells, wherein energy from the light destroys the lenticular epithelial cells in the capsular bag to reduce the growth of epithelial cells that cause posterior capsule opacification after cataract surgery.

9. The system of claim 8, wherein the light is ultraviolet (UV) light and the photosensitizing agent is riboflavin.

10. The system of claim 8, wherein the light source and the optical device deliver the light to the lenticular epithelial cells according to multiphoton excitation.

11. The system of claim 8, wherein the optical device is positioned external to the eye, the optical device directing the light from the light source to the capsular bag.

12. The system of claim 8, wherein the optical device is positioned internally in the capsular bag, the light being transmitted outwardly from the optical device.

13. The system of claim 8, wherein the application system includes a syringe that is configured to hold the photosensitizing agent and deliver the photosensitizing agent into the capsular bag.

14. The system of claim 8, wherein the removal system includes a laser for incising the eye and the capsular bag and for breaking up the first lens for removal from the eye.
